(19) United States
(12) Patent Application Publication
(10) Pub. No.: US 2026/0167372 A1
(43) Pub. Date: Jun. 18, 2026

(54) SEALING SYSTEMS FOR USE WITH PRODUCT BAGS USED IN CRYOGENIC FREEZING AND METHODS OF USE

(71) Applicant: Kite Pharma, Inc., Santa Monica, CA (US)

(72) Inventors: Timothy B. Eyler, Waynesboro, PA (US); Gaurang G. Patel, Frederick, MD (US); Laura B. Radecki, Myersville, MD (US)

(21) Appl. No.: 19/416,861

(22) Filed: Dec. 11, 2025

Related U.S. Application Data
(60) Provisional application No. 63/733,757, filed on Dec. 13, 2024.

(51) Int. Cl. B65B 51/14; A61J 1/10; B65B 61/06; C12N 1/04; C12N 5/00

(52) U.S. Cl. CPC B65B 51/146; A61J 1/10; B65B 61/06; C12N 1/04; C12N 5/546; C12N 5/562

(57) ABSTRACT

A system for sealing a tube extending from a product bag used in cryogenic freezing includes a support having a top surface; a bag guide secured to the support so as to upstand from the top surface, the bag guide including a boundary face; and a tube sealer at least partially upstanding from the top surface of the support, the tube sealer including a first sealing head and a second sealing head and being movable between an open position and a closed position. A spacer arm is movable between a blocking position wherein the spacer arm is disposed between the bag guide and the tube sealer so that the spacer arm at least partially covers the boundary face and an unblocking position wherein the boundary face is openly exposed.

### CROSS-REFERENCE TO RELATED APPLICATIONS

This application claims the benefit under 35 U.S.C. § 119(e) of U.S. Provisional Application No. 63/733,757, filed on Dec. 13, 2024, which is hereby incorporated herein by reference in its entirety for all purposes.

### BACKGROUND OF THE DISCLOSURE

### The Field of the Disclosure

The present disclosure relates to systems for sealing fluid inlet tubes on product bags used for housing and cryogenically freezing biological suspensions and related methods.

### The Relevant Technology

Biological cell suspensions are commonly cryogenically frozen to preserve the viability of the cells for an extended period of time. The process typically entails mixing a cell culture with a cryogenic cell preservation fluid and other desired components to produce a cell suspension. A portion of the cell suspension is then dispensed into a flexible freezer bag through a fluid inlet tube coupled with the freezer bag. A portion of the inlet tube is then sealed closed by forming a welded seal across a portion of the inlet tube. The tube is then cut upstream of the welded seal so that the freezer bag that is housing the cell suspension along with the connected inlet tube and welded seal can be enclosed within a standard cryogenic cassette prior to cryogenic freezing.

When using standard practices, the inlet tube projecting from the freezer bag has an extended length. As a result, it is necessary to bend the inlet tube when placing the freezer bag and inlet tube within the cryogenic cassette. This bending of the inlet tube can produce increased stress on the inlet tube and/or welded seal which in turn increases the risk of failure of the inlet tube and/or welded seal when being placed within the cryogenic cassette, during the cryogenic freezing processing, and/or during movement of the cryogenic cassette. Failure of the inlet tube or welded seal can result in contamination and loss of the cell suspension.

Accordingly, what is needed in the art are methods and systems for sealing a freezer bag housing a biological suspension so as to decrease the risk of failure of the corresponding inlet tube and/or welded seal during placement of the freezer bag into a cryogenic cassette, during freezing of the freezer bag, and/or during manipulation of the cryogenic cassette that is housing the freezer bag.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a top plan view of a bag set used in processing and cryogenically freezing a biological suspension.

FIG. 2 is a top perspective view of a tube sealing system used to seal the fluid inlet tubes of the product bag assemblies shown in FIG. 1.

FIG. 3 is a bottom perspective view of the tube sealing system shown in FIG. 2.

FIG. 4A is a perspective view of a tube sealer of the tube sealing system shown in FIG. 2 in an open position.

FIG. 4B is a perspective view of the tube sealer shown in FIG. 4A in a closed position.

FIG. 5 is a top plan view of one of the product bag assemblies shown in FIG. 1 having seals applied to the inlet tube thereof prior to placement on tube sealing system.

FIG. 6 is a perspective view of the product bag assembly shown in FIG. 5 mounted on the tube sealer system shown in FIG. 2 with a spacer arm thereof in a raised position.

FIG. 7 is a perspective view of the product bag assembly shown in FIG. 6 mounted on the tube sealer system with the spacer arm in a closed position.

FIG. 8 is a top plan view of the product bag assembly after being removed from the tube sealer system in FIG. 7.

FIG. 9 is a perspective view of a cryogenic cassette assembly configured to receive the product bag assembly shown in FIG. 8.

FIG. 10 is a perspective view of the product bag assembly shown in FIG. 8 received within the cryogenic cassette assembly shown in FIG. 9.

FIG. 11 is a top plan view of the assembly shown in FIG. 10 with the cryogenic cassette in a closed and locked position.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12A” and “12B.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

In general, the present disclosure relates to methods and systems for effectively sealing closed an inlet tube of a product bag assembly, particularly where the product bag assembly is configured for housing a cell suspension and being placed within a cryogenic cassette for cryogenic freezing.

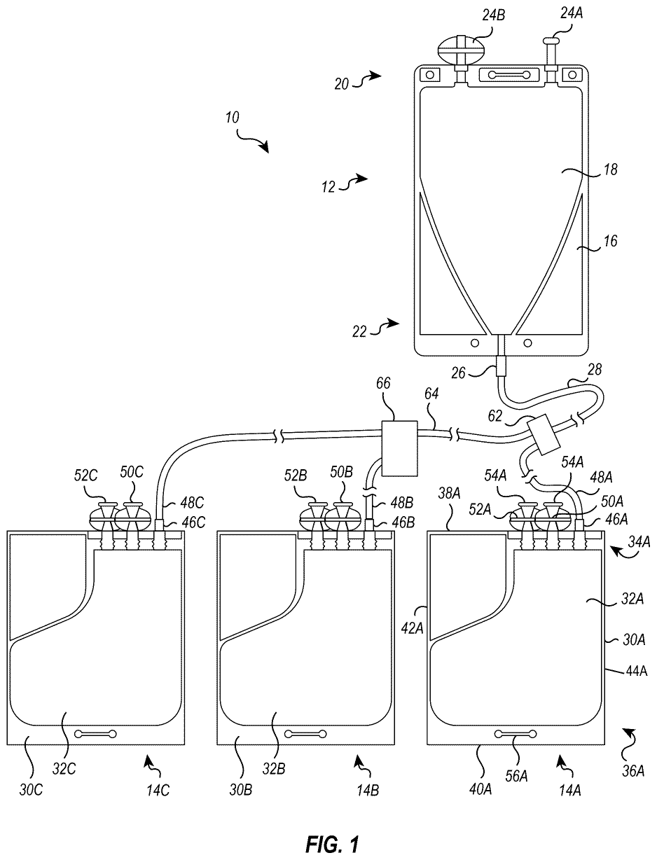

Depicted in FIG. 1 is one embodiment of bag set 10 designed for processing biological fluids such as biological cell suspensions. Bag set 10 comprises a processing bag assembly 12 fluid coupled with a plurality product bag assemblies 14A, 14B, and 14C. Processing bag assembly 12 comprises a processing bag 16 that bounds a compartment 18 that extends between an upper end 20 and an opposing lower end 22. In one embodiment, processing bag 16 comprises a flexible, collapsible bag. For example, processing bag 16 can be comprised of one or more sheets of a flexible, water impermeable polymeric film. The polymeric film can commonly have a thickness that is less than 2 mm, 1 mm, 0.5 mm, 0.2 mm, 0.05 mm, or 0.02 mm or be in a range between any two of the foregoing. Other thicknesses can also be used. The film is sufficiently flexible that it can be rolled into a tube without plastic deformation and can be folded over an angle of at least 180° or 360° without plastic deformation. The material can be approved for direct contact with living cells and be capable of maintaining a solution sterile. In one embodiment, processing bag 16 can comprise a two-dimensional pillow style bag. For example, two sheets of polymeric film can be placed in overlapping relation and the two sheets bound together at their peripheries to form compartment 18. Other methods can also be used. In alternative embodiments, processing bag 16 can comprise a three-dimensional bag.

Processing bag assembly 12 also includes one or more ports disposed at upper end 20. In the depicted embodiment, ports 24A and 24B are shown that communicate with compartment 18. Ports 24A and 24B can be used for delivering fluid into and/or removing fluid from compartment 18. Disposed at lower end 22 is an outlet port 26 that communicates with compartment 18 and has an outlet tube 28 fluid coupled therewith. A tube can also be permanently or removably coupled with port 24A and/or port 24B. As discussed below, outlet tube 28 is in fluid communication with each of product bag assemblies 14A, 14B, and 14C.

Product bag assembly 14A comprises a product bag 30A bounding a compartment 32A extending between an upper end 34A and an opposing lower end 36A. Upper end 34A terminates at an upper edge 38A while lower end 36A terminates at a lower edge 40A. Upper edge 38A and lower edge 40A each extend laterally to opposing side edges 42A and 44A. Although not required, in one embodiment upper edge 38A and lower edge 40A can each extend linearly between opposing side edges 42A and 44A. Product bag 30A can have the same properties, be made of the materials, and have the same configuration as discussed above with regard to processing bag 16. For example, product bag 30A can comprise a flexible, collapsible, two-dimensional pillow style bag comprised of one or more sheets of a flexible, water impermeable polymeric film. However, in contrast to processing bag 16, which can but does not necessarily need to be able to withstand cryogenic freezing, product bag 30A is designed to withstand cryogenic freezing.

Disposed at upper end 34A of product bag 30A is a tubular stem 46A that communicates with compartment 32A. In one embodiment stem 46A passes through upper edge 38A, i.e., is sealed between overlapping sheets of film that are bound together to form upper edge 38A. Secured to stem 46A is a flexible fluid inlet tube 48A that projects away from product bag 30A and is in fluid communication with outlet tube 28. Fluid inlet tube 48A is typically comprised of a polymeric material. Also disposed at upper end 34A are ports 50A and 52A that communicate with compartment 32A. Ports 50A and 52A are sealed closed but can be opened to enable selective withdrawal of fluid contained within compartment 32A. Ports 50A and 52A can be adjacently disposed, extend through upper edge 38A, and each terminate at a terminal end 54A. Each of port 50A, port 50B, and stem 46A can be disposed within a common plane and extend in parallel alignment. In one embodiment, ports 50A and 52A can each comprise a spike port. Other types of ports can also be used. In alternative embodiments, ports 50A and 50B can each be the same type of port or can be different types of ports. Depending on intended use, one of ports 50A or 50B can be eliminated or an additional port added. An optional opening 56A centrally extends through lower end 36A of product bag 30A spaced apart from compartment 32A. Opening 56A allows for easy hanging or supporting of product bag assembly 14A from lower end 36A.

Each of product bag assemblies 14B and 14C can have the same configuration, properties, alternatives, and be made of the same materials as product bag assembly 14A. As such, all of the discussion herein with regard to product bag assembly 14A is also applicable to product bag assemblies 14B and 14C, unless otherwise indicated. In addition, like elements between product bag assemblies 14A, 14B, and 14C are identified by like reference numbers except that the reference numbers for product bag assembly 14B and 14C include the suffix “B” and “C”, respectively.

In the depicted embodiment, a first manifold 62 is used to fluid couple together outlet tube 28 of processing bag assembly 12 and inlet tube 48A of product bag assembly 14A. A tube section 64 extends between an outlet side of first manifold 62 and an inlet side of a second manifold 66. In turn, inlet tubes 48B and 48C of product bag assemblies 14B and 14C, respectively, fluid couple with an outlet side of second manifold 66. As a result, by selectively clamping or sealing combinations of inlet tubes 48A-48C, fluid can be directed from processing bag assembly 12 to each product bag assembly 14A-14C, either under the force of gravity or with the use a peristaltic pump or some other mechanism.

It is appreciated that in alternative embodiments that bag set 10 can have a variety of different configurations. For example, in contrast to having three product bag assemblies, processing bag assembly 12 can be fluid coupled with 1, 2, 3, 4 or more product bag assemblies 14. In addition, depending on intended use, some product bag assemblies can have a different configuration than others. In other embodiments, processing bag assembly 12 can be replaced with a rigid container or some or reservoir or fluid source. Other alternatives can also be used. One example of a bag set that can be used at bag set 10 is the FINIA Fill and Finish System, 3 Bag Set, available from Fisher Scientific. Comparable bags sets are also available from Origen Biomedical, Inc. In contrast to starting with a bag set having multiple bags, the tube sealing system of the present disclosure, as discussed below, can be used with a single product bag assembly, such as a CRYOSTORE freezing bag available from Origen Biomedical, Inc., that is not integrally formed with other bags or containers but can be selectively fluid coupled with another bag or container.

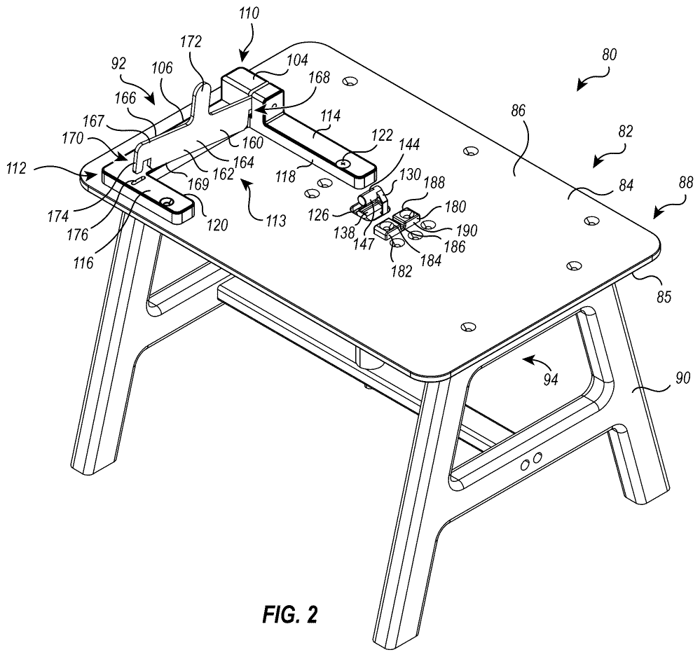

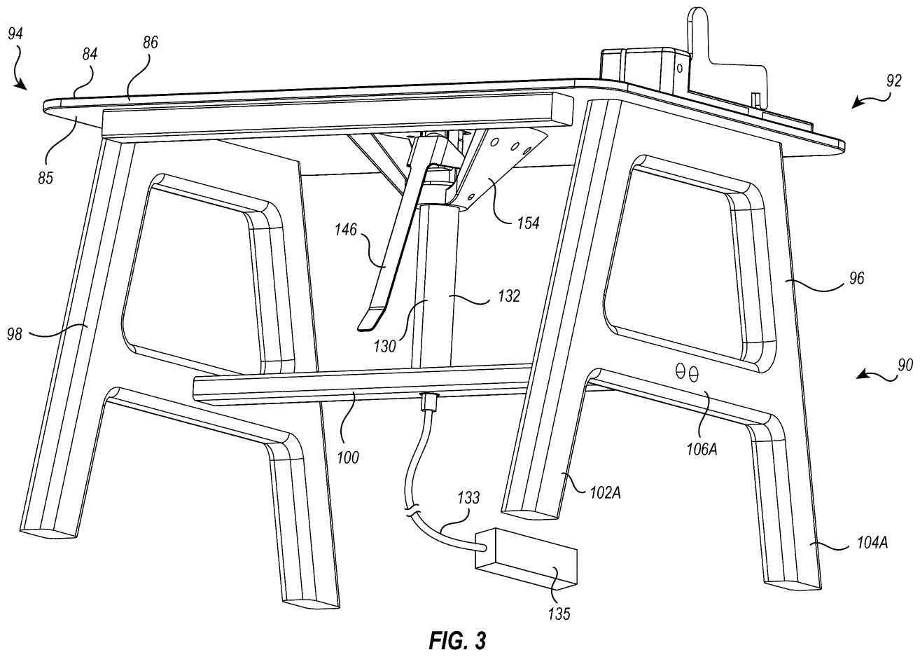

Depicted in FIG. 2 is one embodiment of a tube sealing system 80 that, as discussed below in further detail, can be used to seal closed the inlet tubes 48 of product bag assemblies 14. Tube sealing system 80 comprises a support 82 having a top surface 84. In the depicted embodiment, support 82 can comprise a tabletop 86 having top surface 84. More specifically, in one embodiment support 82 can comprise a table 88 that includes tabletop 86 supported on a leg assembly 90. With reference to FIGS. 2 and 3, tabletop 86 includes top surface 84 and an opposing bottom surface 85 that each extend between a first end 92 and an opposing second end 94. Top surface 84 is typically planar and in the depicted embodiment has a square or rectangular configuration with rounded corners. Other configurations can also be used.

Leg assembly 90 includes a first leg support 96 downwardly projecting from tabletop 86 at first end 92, a second leg support 98 downwardly projecting from tabletop 86 at second end 94, and a brace 100 centrally extending between first leg support 96 and second leg support 98. More specifically, first leg support 96 comprises a first leg 102A, a second leg 104A, and a side stretcher 106A extending therebetween. Likewise, second leg support 98 comprises a first leg 102B, a second leg 104B, and a side stretcher 106B extending therebetween. Brace 100 centrally extends between side stretcher 106A and side stretcher 106B. It is appreciated that leg assembly 90 and leg supports 96 and 98 can have a variety of different configurations, as is known in the art.

Tube sealing system 80 further comprises a bag guide 104 secured to support 82/tabletop 86 at or toward first end 92 so as to upstand from top surface 84. Bag guide 104 includes a back brace 106 having a boundary face 108 that extends between a first end 110 and an opposing second end 112. Boundary face 108 faces toward second end 94 of tabletop 86. In one embodiment, boundary face 108 upwardly projects perpendicular to top surface 84 of tabletop 86. Bag guide 104 can also include a first side brace 114 projecting from first end 110 of back brace 106 toward second end 94 of tabletop 86 and/or a second side brace 116 projecting from second end 112 of back brace 106 toward second end 94 of tabletop 86. In one embodiment bag guide 104 has a U-shape configuration that bounds a slot 113. Side braces 114 and 116 can outwardly project perpendicular to back brace 106 and/or boundary face 108. More specifically, first side brace 114 includes a first guide face 118 facing toward slot 113/second side brace 116 while second side brace 116 includes a second guide face 120 facing toward slot 113/first side brace 114. First guide face 118 and second guide face 120 can thus face opposingly and be disposed parallel to each other while each being perpendicular to boundary face 108. Faces 108, 118, and 120 can also have a U-shaped configuration and bound slot 113 disposed above top surface 84.

Bag guide 104 can be integrally formed with or be permanently secured to tabletop 86. More commonly, however, bag guide 104 is typically removably secured to tabletop 86 so that it can be easily replaced if damaged or can be switched out for a different size or configuration. Bag guide 104 can be removably secured to tabletop 86 by using fasteners 122, such as screws, bolts, pins, or the like that pass down through bag guide 104 and engage with tabletop 86. Other mechanisms can also be used. Bag guide 104 can also have a variety of other configurations. For example, first side brace 114 and/or second side brace 116 can comprise members that are separate and discrete from back brace 106 and that are independently secured to tabletop 86. In this regard, first side brace 114, second side brace 116, and back brace 106 can be spaced apart from either other or can be disposed directly adjacent to each other.

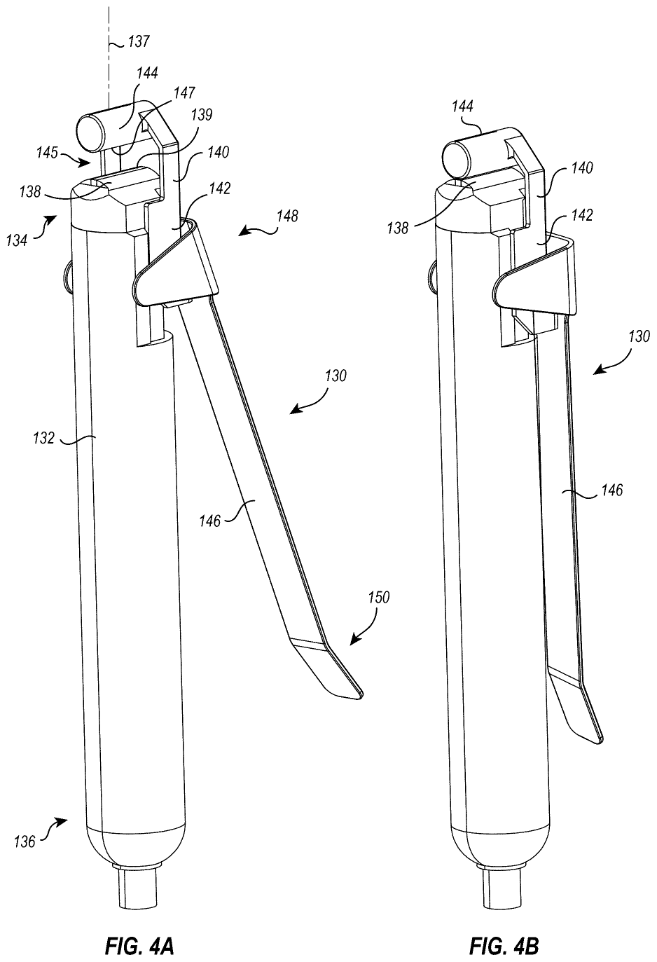

Tabletop 86 includes an opening 126 that passes down through tabletop 86 between top surface 84 and bottom surface 85 and that is aligned with slot 113. Tube sealing system 80 also includes a tube sealer 130 that partially projects through opening 126 so as to upstand from top surface 84. Turning to FIGS. 4A and 4B, in one embodiment tube sealer 130 comprises a radio frequency tube sealer that includes an elongated body 132 having an upper end 134 and an opposing lower end 136 with a central longitudinal axis 137 extending along the length thereof. Upper end 134 terminates at a rounded first sealing head 138 that is elongated. An electrical cable 133 (FIG. 3) couples with lower end 136 of body 132 and is connected to a controller 135. When activated, controller 135 and body 132 operate as a radio frequency generator. Controller 135 can also include a power source, such as a battery, or can be connected to an electrical outlet for powering the radio frequency generator.

Tube sealer 130 also includes a carriage assembly 140 that includes a carriage base 142 having a second sealing head 144 mounted thereon. Carriage assembly 140/carriage base 142 is slidably mounted on upper end 134 of body 132. Specifically, carriage base 142 and upper end 134 of body 132 engage by liner tracks that allow carriage base 142 to slide linearly along body 132/longitudinal axis 137. Second sealing head 144 also has an elongated rounded surface and in the depicted embodiment has a cylindrical configuration.

Tube sealer 130 further includes an elongated handle 146 having an upper end 148 and an opposing lower end 150. Upper end 148 is hingedly coupled with upper end 134 of body 132 and is engaged with carriage assembly 140 so that tube sealer 130 can be selectively moved between an open position and a closed position. In the open position, as shown in FIG. 4A, lower end 150 of handle 146 is spaced apart from body 132 and second sealing head 144 is spaced apart from first sealing head 138 so that a gap 145 is formed therebetween. To move tube sealer 130 to the closed position, an operator manually or mechanically pulls lower end 150 of handle 146 toward body 132. In so doing, handle 146 pivots relative to body 132 and carriage assembly 140 slides downward along body 132 toward lower end 136. In so doing, sealing heads 138 and 144 are drawn together.

Durning operation, as discussed below, a portion of inlet tube 48 (FIG. 1) is position between sealing heads 138 and 144 when in the open position. As tube sealer 130 is moved to the closed position, inlet tube 48 is compressed closed between sealing heads 138 and 144. Specifically, sealing head 138 has a rounded engaging surface 139 that directly engages with inlet tube 48 and sealing head 144 has a opposing rounded engaging surface 147 that directly engages with inlet tube 48 when tube sealer 130 is moved to the closed position. Concurrently, as handle 146 is moved to the closed position, high-frequency radio waves are generated within body 132/controller 135 and are directed to inlet tube 48 compressed between sealing heads 138 and 144. The radio waves heat the plastic tube material causing it to melt and fuse together under the applied pressure, thereby forming a weld seal across inlet tube 48. As handle 146 is released, a spring resilient returns handle 146/tube sealer 130 to the open position. The radio waves can automatically be deactivated based on movement of handle 146, termination of a time period, change in applied pressure, or other mechanism. Deactivation of the radio waves allows inlet tube 48 and the resulting weld seal to be removed from between sealing heads 138 and 144.

One example of a radio frequency tube sealer that can be used as tube sealer 130 is the SEBRA Hand-Held RF Tube Sealing System available from Vante Biopharma/Sebra, Models 1105 and 2380. In contrast to tube sealer 130 being a radio frequency sealer, tube sealer 130 can also function to heat inlet tube and form the weld seal by thermal energy, e.g., directly heating sealing heads 138 and 144, sonic welding, or other welding techniques used to weld closed plastic tubes.

During assembly, tube sealer 130 is mounted so that second sealing head 144 projects through opening 126 and extends above top surface 84 of tabletop 86. Tube sealer 130 is typically positioned so that first sealing head 138 or engaging surface 139 thereof that engages inlet tube 48 is flush to top surface 84. In alternative embodiments, engaging surface 139 is typically less than 2 cm, 1.5 cm, 1 cm, 0.5 cm. 0.2 cm, or 0.1 cm above or below top surface 84. This positioning of engaging surface 139 helps with optimizing the formation and positioning of the weld seal on inlet tube 48, as discussed below. Handle 146 is disposed below tabletop 86. A bracket 154 can be mounted to bottom surface 85 of tabletop 86 to help secure upper end 134 of body 132 in the proper position while brace 100 can be used to secure and support lower end 136 of body 132. Table top 86 can be opaque or translucent. One benefit of making tabletop 86 translucent is that it enables an operator to easily locate and operate handle 146 by looking through tabletop 86 during operation.

Returning to FIG. 2, tube sealing system 80 also includes a spacer arm 160 that is movable between a blocking position and an unblocking position. In the blocking position, spacer arm 160 is disposed between back brace 106 of bag guide 104 and tube sealer 130 so that spacer arm 160 at least partially covers boundary face 108. In the unblocking position, spacer arm 160 is moved so that boundary face 108 is openly exposed. More specifically, in one embodiment spacer arm 160 comprises a plate 162 having a front face 164 and an opposing back face 166 that laterally extend between a top edge 167 and an opposing bottom edge, and that each longitudinally extend between a first end 168 and an opposing second end 170. Plate 162 commonly has a thickness extending between front face 164 and back face 166 that is typically larger than 0.2 cm, 0.3 cm, 0.4 cm or 0.5 cm or is commonly in a range between 0.2 cm and 1 cm with between 0.3 cm and 0.8 cm and between 0.3 cm and 0.5 cm being more common. Other dimensions can also be used depending on application. Front face 164 and/or back face 166 are commonly planar and can be disposed in parallel alignment. In the depicted embodiment, first end 168 is hingedly mounted to bag guide 104 and, more specifically, to first side brace 114. An optional tab 172 upwardly projects from top edge 167 of plate 162 and can be used to easily grasp and pivot plate 162/spacer arm 160 between the blocking position and the unblocking position.

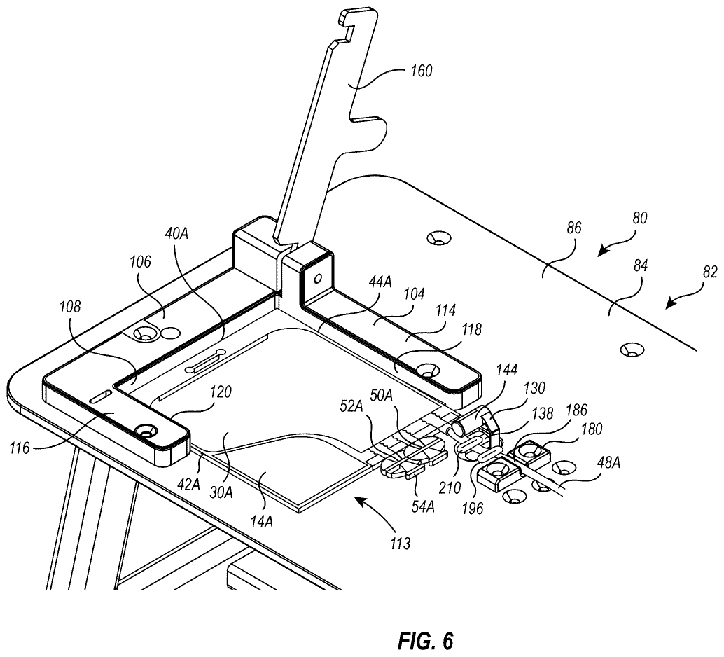

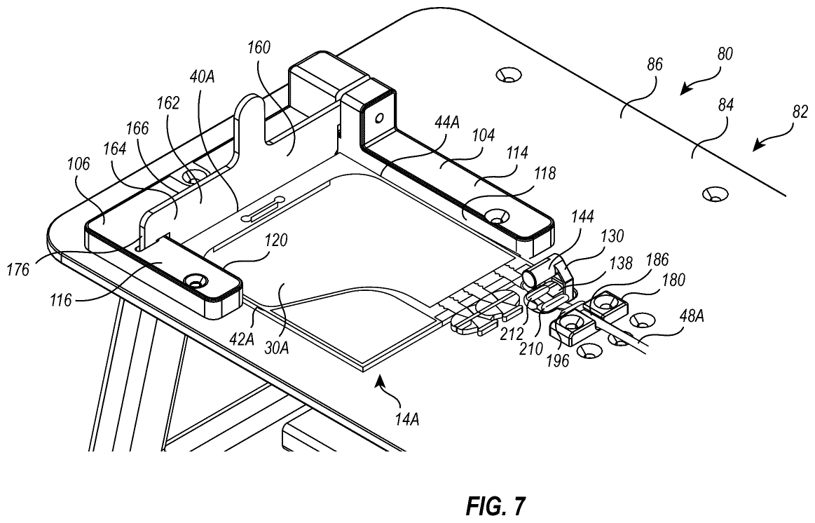

With reference to FIGS. 2 and 7, in the blocking position plate 162/spacer arm 160 is pivoted so that bottom edge 169 of plate 162 rests directly on top surface 84 of table top 86 while back face 166 is disposed directly against or adjacent to boundary face 108 of back brace 106 so as to at least partially cover boundary face 108. Spacer arm 160 can include a downwardly projecting finger 174 formed at second end 170 of plate 162 while a corresponding slot 176 is recessed into second side brace 116. Finger 174 is configured to be received within slot 176 when spacer arm 160 is in the blocking position. This helps to ensure that both opposing ends of spacer arm 160 are properly positioned relative to back brace 106/boundary face 108 when in the blocking position. With reference to FIGS. 2 and 6, when spacer arm 160 is moved to the unblocking position, spacer arm 160 is pivoted or otherwise moved so that back brace 106/boundary face 108 is openly exposed and thus freely accessible from slot 113. Thus, as shown in FIG. 6, spacer arm 160 is pivoted relative to bag guide 104 so as to be upstanding away from boundary face 108.

It is appreciated that spacer arm 160 can have a variety of different configurations and be moved in a variety of different ways between the blocking position and an unblocking position. For example, in contrast to being pivotably mounted to bag guide 104, spacer arm 160 could be pivotably mounted to tabletop 86 outside of bag guide 104 with a slot and/or notch formed on spacer arm 160 or bag guide 104 that allows spacer arm 160 to pivot between the blocking position and the unblocking position. In one alternative where first side brace 114 is formed separate and discrete from back brace 106, spacer arm 160 can be pivotably mounted to first side brace 114 or could be pivotably mounted to tabletop 86 at a location between first side brace 114 and back brace 106.

In another alternative, spacer arm 160 can be freely removable from tabletop 86 and bag guide 104. For example, a notch can be formed on each of side braces 114 and 116 adjacent to boundary face 108. The notches can be configured to receive the opposing ends of spacer arm 160. As such, spacer arm 160 can be lowered into the notches to be in the blocking position and removed from bag guide 104 to be in the unblocked position. In yet another alternative, a clamp, bolt, clip or other fastener can be used to removably hold spacer arm 160 against boundary face 108.

Returning to FIG. 2, tube sealing system 80 also includes a tube guide 180 upstanding from the top surface 84 of support 82/tabletop 86 on a side of tube sealer 130 opposite of bag guide 104. Tube guide 180 includes a base 182 having a top face 184 with a slot 186 recessed into and across top face 184 from a front face 188 to an opposing back face 190. Tube guide 180 can be permanently fixed to or removably secured to support 82/tabletop 86 so that slot 186 is aligned with sealing heads 138 and 144 of tube sealer 130 and gap 145 therebetween. Slot 186 is configured so that a desired tube, e.g., inlet tube 48, can be manually wedged therein so as to securely hold the tube in a linear orientation while the weld seal is being formed by tube sealer 130. Thereafter, the tube can be easily removed from slot 186 by simply pulling on the tube. By making tube guide 180 removable from tabletop 86, tube guide 180 can be removed and replaced with a different tube guide 180 having a slot 186 of a different size to accommodate different sizes of tubes. Likewise, tube guide 180 can be moved to different locations on tabletop 86 to account for different sizes of bag assemblies, as discussed below. It is appreciated that tube guide 180 can have a variety of different configurations. For example, in alternative embodiments, tube guide 180 can comprise a clamp that engages the tube, a restraint that is placed over the tube and secured to tabletop 86, or a restraint secured to tabletop 86 that folds over the tube so that the tube is sandwiched and secured therein. Other configurations can also be used. However, the depicted tube guide 180 has unique benefits in that it is simple in design, easy to use, and can quickly secure and release the tube.

In the depicted embodiment, support 82 is disclosed as a table. In alternative embodiments, however, support 82 can comprise a shelf, cabinet, dresser, desk, work bench, or other structure that includes a top surface on which bag guide 104, spacer arm 160, tube sealer 130, and tube guide 180 can be disposed.

Returning to FIG. 1, during use, a biological suspension is dispensed into or produced within compartment 18 of processing bag assembly 12. The biological suspension can comprise a cell culture combined with at least one or more of a sterile water, buffer, reagent, and/or cryopreservation fluid. Other components can also be added. The cell culture can comprise plant cells, animal cells, or human cells. For example, the cell culture can comprise blood cells and, more specifically, white blood cells. The cell culture will typically have been grown and concentrated, such as in a bioreactor, prior to being passed into processing bag assembly 12. The other components of the suspension can be combined with cell culture prior to and/or after delivering the cell culture into processing bag assembly 12. The biological suspension is typically mixed within processing bag assembly 12 and can be temperature regulated.

In one method of use, bag set 10 is first mounted on a filling system prior to use. The filling system can include discrete stations where each of processing bag assembly 12 and product bag assembly 14 are separately secured. The filling system also typically has a mechanism for selectively pinching tubes 28 and 48 closed and/or sealing tubes 28 and 48 closed. For example, the filling system can include radio frequency tube sealers or any of the other tube sealers discussed above. In one embodiment, processing bag assembly 12 is secured higher than product bag assemblies 14 so that the biological suspension can flow under gravity to each of product bag assemblies 14. In other embodiments, a peristaltic pump can be placed on outlet tube 28 and/or a plate can be selectively pressed against processing bag assembly 12 to help drive and control fluid flow. Other conventional mechanisms for facilitating fluid flow from processing bag assembly 12 to product bag assemblies 14 can also be used. One example of a filling system that can be used as discussed above is the FINIA Fill and Finish System available from Terumo Bood and Cell Technologies (Terumo BCT).

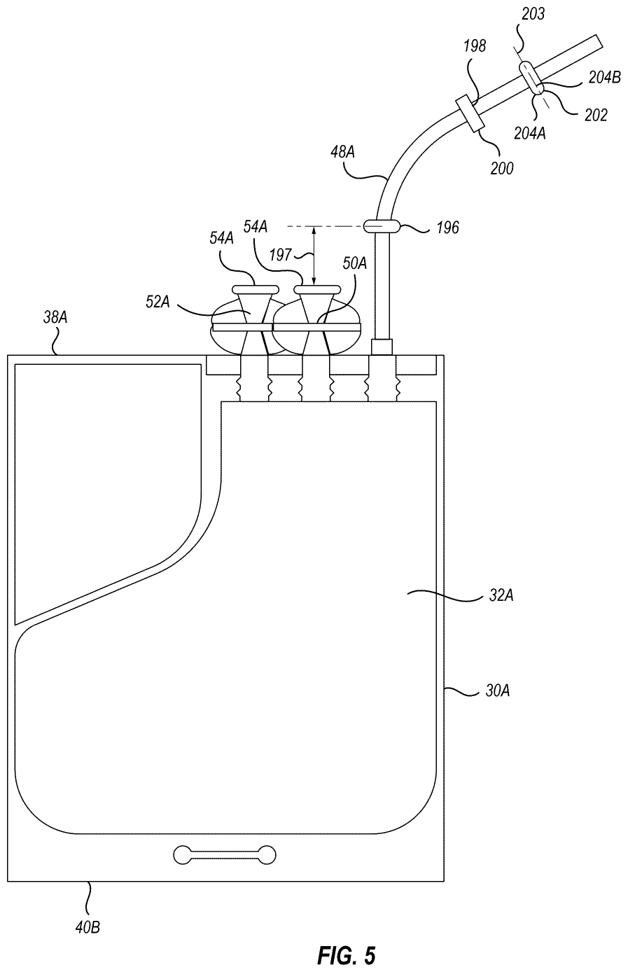

Once bag set 10 is secured to the filling system and the biologic suspension is within processing bag assembly 12 and mixed, as desired, the filling system can be configured to deliver the biological suspension into product bag assembly 14A. Once a predefined quantity of the biological suspension is within product bag assembly 14A, the flow from processing bag assembly 12 is stopped and a weld seal 196, as shown in FIG. 5, is formed across inlet tube 48A so as to seal closed the passage extending therethrough. Because of the way conventional filling systems are configured, weld seal 196 is formed at a spaced apart position from the terminal end 54A of ports 50A and 52A. That is, when ports 50A and 52A and inlet tube 48A are each extending linearly from product bag 30A/upper edge 38A in parallel alignment, a distance 197 between the terminal end face 54A of ports 50A and 52A and a center of weld seal 196, as measured along inlet tube 48A, is at least 0.5 cm, 1 cm, 1.2 cm, 1.5 cm, 2 cm, 2.5 cm or 3 cm or is in range between any two of the foregoing.

Although not required, in one embodiment of the present disclosure, a second seal is formed across inlet tube 48A before product bag assembly 14A is separated from processing bag assembly 12. Accordingly, either prior to or after forming weld seal 196, a seal 198 is formed across inlet tube 48A upstream from the location for weld seal 196. Seal 198 can comprise a clamp seal. That is, a clamp 200 can be placed over inlet tube 48A so as to form seal 198 that seals inlet tube 48A closed thereat. By way of example, clamp 200 can comprise a hose clamp, hemostat, or other clamping device. Clamp 200 can be removable or permanently fixed. In one embodiment, seal 198 is formed at a linear distance along inlet tube 48A that is at least 5 cm, 6 cm, 7 cm, 8 cm, 10 cm from product bag 30A/upper edge 38A or is located at a range between any two of the foregoing. Always having at least two seals on inlet line 48A helps to ensure that the suspension within product bag 30A has not been contaminated and allows for improved certification of the sterility of the suspension.

Following the formation of weld seal 196 and seal 198, a portion of inlet tube 48A, typically upstream from seal 198, is placed between sealing heads 138 and 144 of tube sealer 130. Tube sealer 130 is then activated so as to form a test weld seal 202 across inlet tube 48A upstream of seal 198. By using tube sealer 130 to form test weld seal 202, the operator is able to visually inspect test weld seal 202 to ensure tube sealer 130 is properly functioning before the final weld seals are produced, as discussed below. In an alternative embodiment, test weld seal 202 could be formed on a separate tube for ensuring proper operation of tube sealer 130. However, forming test weld seal 202 on inlet tube 48A both helps to further protect against unwanted contamination of the suspension and forms an area for cutting inlet tube 48A. That is, once test weld seal 202 is formed and properly inspected, product bag assembly 14A can be separated from processing bag assembly 12/bag set 10, by laterally cutting through a center 203 of test weld seal 202. As a result, a first portion 204A of weld seal 202 remains formed at the now terminal end of inlet tube 48A so as to seal inlet tube 48A closed thereat while a second portion 204B of weld seal 202 remains formed on the severed end of inlet tube 48A so as to seal the inlet tube 48A closed thereat. Seal portions 204A and 204B help to maintain sterility of the biological suspension in bag assembly 14A and in the remainer of bag set 10. In addition, seal portions 204A and 204B help prevent the leaking out of any suspension from inlet tube 48A during cutting inlet tube 14A. The cutting can be performed with scissors, knife, or other cutting tools.

Turing to FIG. 6, with product bag assembly 14A separated from processing bag assembly 12/bag set 10, product bag assembly 14A can be placed on top surface 84 of table top 86/support 82 so as to be received with slot 113. Specifically, spacer arm 160 is moved to the unblocked position and product bag assembly 14A is positioned within slot 113 so that lower edge 40A of product bag 30A butts directly against boundary face 108 of back brace 106. Although not required, bag guide 104 can also be configured so that side edge 42A and/or side edge 44A butt directly against or are disposed directly adjacent to guide face 120 and guide face 118, respectively. The engagement of product bag 30 with guide faces 118 and 120 helps to ensure that product bag 30A/product bag assembly 14A is properly positioned and orientated with slot 113.

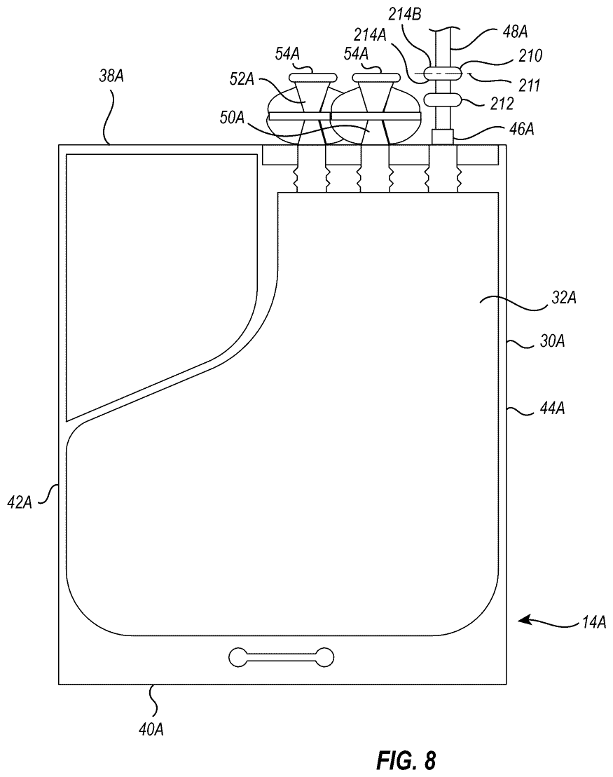

With product bag 30A/product bag assembly 14A so positioned, inlet tube 48A is passed through gap 145 between sealing heads 138 and 144 (FIG. 4A). Inlet tube 48A is then straightened and secured within slot 186 of tube guide 180. In this position, inlet tube 48A may be resting directly on sealing head 138/engagement surface 139 and weld seal 196 is typically located between tube guide 180 and tube sealer 130. Inlet tube 48A will typically want to resiliently curve or bend. Tube guide 180 thus functions, in part, to keep inlet tube 48A straight and maintained centrally between sealing heads 138 and 144 of tube sealer 130 so that a further weld seal can be properly formed on inlet tube 48A. Specifically, with product bag assembly 14A mounted on tube sealing system 80, as discussed above with regard to FIG. 6, the operator moves tube sealer 130 from the open position to the closed position, as discussed above, so as to produce a weld seal 210 on inlet tube 48A (FIGS. 6 and 8) that is aligned with or generally aligned with the terminal ends 54A of ports 50A and 52A. More specifically, when ports 50A and 52A and inlet tube 48A are each extending linearly from product bag 30A/upper edge 38A in parallel alignment, a plane extending through a lateral central axis 211 of weld seal 210 and disposed perpendicular to inlet tube 48A is aligned with terminal ends 54A of ports 50A and 52A or is within at least +/−5 mm, 3 mm, 2 mm, or 1 mm from the terminal ends 54A of ports 50A and 52A. Commonly, weld seal 210 is positioned so that the above plane passing through lateral central axis 211 is disposed between terminal end 54A and upper edge 38A and is commonly spaced apart from terminal end 54A by at least 1 mm, 2 mm, 3 mm, or 5 mm, depending on intended use, or is in a range between any two of the foregoing.

Turing to FIG. 7, once weld seal 210 is formed, product bag assembly 14A is moved forward within slot 113 of bag guide 104. Spacer arm 160 is then moved to the blocking position. Product bag assembly 14A is manually positioned so that that lower edge 40A of product bag 30A/product bag assembly 14A now butts directly against front face 164 of spacer arm 160. Again, side edges 42A and/or 44A can butt directly against or be disposed directly adjacent to guide face 120 and guide face 118, respectively, for proper positioning of product bag 30A/product bag assembly 14A. Inlet tube 48A is manually adjusted to extend linearly from stem 46A/product bag 30A and is secured within tube guide 180. Inlet tube 48A passes between sealing heads 138 and 144 of tube sealer 130 with weld seals 210 and 196 now being disposed between tube sealer 130 and tube guide 180. Once so positioned, the operator again activates tube sealer 130 so that tube sealer 130 is moved from the open position to the closed position, as discussed above, thereby producing a weld seal 212 across inlet tube 48A (FIGS. 7 and 8) at a location between weld seal 210 and stem 46A/product bag 30A.

In one embodiment, the linear spacing between weld seals 210 and 212 and the linear spacing between weld seal 212 and the terminal end of stem 46A is at least 2 mm, 3 mm, 4 mm, 5 mm, 6 mm, 8 mm, or 10 mm or is in a range between any two of the foregoing. This spacing is useful to help ensure the proper formation of weld seal 212. The spacing between weld seals 210 and 212 can be the same or different than the spacing between weld seal 212 and the terminal end of stem 46A. In view of the foregoing, it is appreciated that the thickness of spacer arm 160 correlates to the spacing between weld seals 210 and 212 and that the use of spacer arm 160 provides a simple mechanism for accurately and precisely spacing weld seals 210 and 212. Once both of weld seals 210 and 212 are formed, weld seal 210 is cut laterally along central axis 211 so that a portion 214A, now at a terminal end of inlet tube 48A extending from product bag 32A, seals the end of fill tube 48A closed. A portion 214B of weld seal 210 also remains on the end of the now separated portion of inlet tube 48A, thereby ensuring the sterility of the suspension with product bag 30A and preventing any fluid leakage from inlet tube 48A. Here again it is noted that at least two seals, e.g., weld seal 212 and portion 214B of weld seal 210, can always be maintained on inlet tube 48A when inlet tube 48A is being cut so as to preserve sterilization and enable certification.

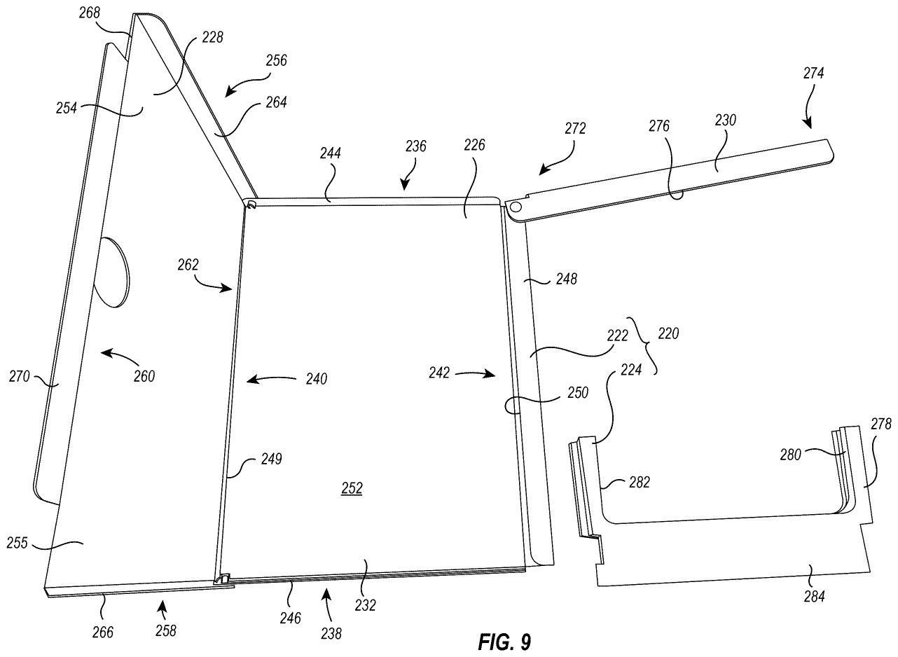

Once product bag assembly 14A is properly sealed and trimmed, as discussed above with regard to FIGS. 7 and 8, product bag assembly 14A is then placed within a cryopreservation cassette and then cryogenically frozen. Specifically, depicted in FIG. 9 is one embodiment of cryopreservation cassette assembly 220 that includes a cassette 222 and a stabilizer 224. Cassette 222 includes a base 226, a cover 228 hingedly mounted to base 226, and a locking arm 230 hingedly mounted to base 226. Base 226 includes bottom panel 232 having a planar interior face 234 longitudinally extending between a first end 236 and an opposing second end 238 and laterally extending between a first side 240 and an opposing second side 242. A first lip 244 outwardly projects along the width of bottom panel 232 at first end 236 while a second lip 246 outwardly projects along the width of bottom panel 232 at second end 238. Lips 244 and 246 can outwardly project orthogonal to bottom panel 232 and can be disposed in parallel alignment. A third lip 249 extends along the length of first side 240 of bottom panel 232 and can also outwardly project orthogonal to bottom panel 232. A shoulder 248 extends along the length of second side 242 of bottom panel 232 and can outwardly project orthogonal to bottom panel 232. Shoulder 248 has a U-shaped transverse cross section that bounds a recess 250 facing toward bottom panel 232/third lip 249. Base 226 at least partially bounds a cavity 252 in which product bag assembly 14A is received and which is at least partially encircled by lips 244, 246, 249, and shoulder 248.

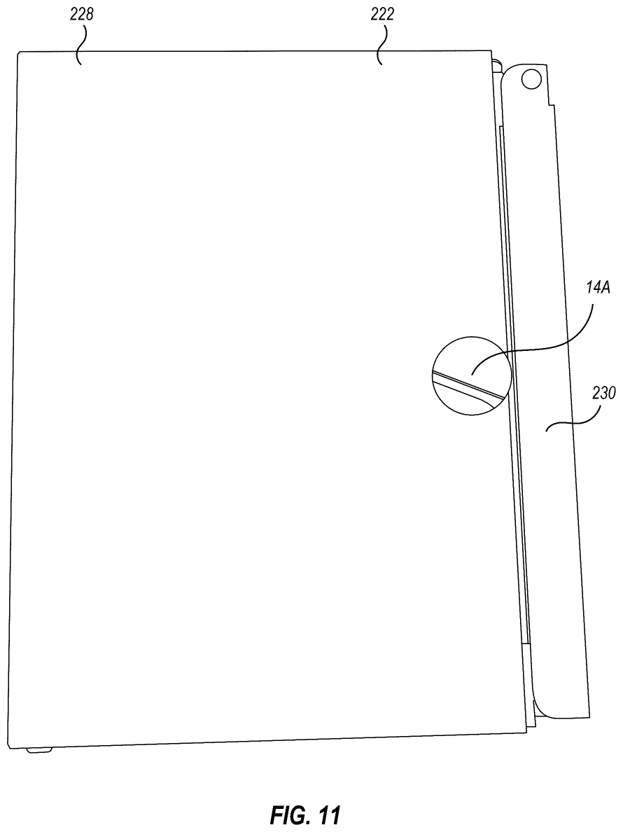

Cover 228 includes a top panel 254 having a planar interior face 255 longitudinally extending between a first end 256 and an opposing second end 258 and laterally extending between a first side 260 and an opposing second side 262. A first lip 264 outwardly projects along the width of top panel 254 at first end 256 while a second lip 266 outwardly projects along the width of top panel 254 at second end 258. Lips 264 and 266 can outwardly project orthogonal to top panel 254 and be disposed in parallel alignment. First side 260 terminates at a side edge 268. A planar locking tab 270 outwardly projects from side edge 268 in the same plane as top panel 254. Lips 244 and 264 are hingedly coupled together while lips 244 and 246 are hingedly coupled together so as to allow cover 228 to pivot relative to base 226 between an open position, as shown in FIG. 9 wherein cavity 252 is openly exposed, and a closed position, as shown in FIG. 11, wherein cover 228/top panel 254 extends over base 226/ bottom panel 232 so as to substantially enclose cavity 252. In the closed position, locking tab 270 is disposed on top of shoulder 248.

Locking arm 230 has a U-shaped transverse cross section that longitudinally extends between a first end 272 and an opposing second end 274. First end 272 of locking arm 230 is hingedly coupled to a first end of shoulder 248 while opposing second end 274 is freely disposed. Locking arm 230 bounds a channel 276 that is configured to receive shoulder 248. Specifically, when cover 228 is in the closed position, locking arm 230 can be pivoted so that both shoulder 248 and adjacent locking tab 270 are received within channel 276 of locking arm 230, thereby securing cover 228 to base 226 in the closed position.

Optional stabilizer 224 includes a U-shaped body 278 having an interior surface with a groove 282 extending along the length thereof. A tab 284 outwardly projects from a bottom of body 278. Stabilizers 224 can come in different configurations having tabs 284 of different lengths depending on the size of bag being received within cavity 252 of cassette 222. That is, one function of stabilizer 224 is to occupy extra space within cavity 252 to help limit movement of product bag assembly 14A within cavity and thereby limit the risk of damage to bag assembly 14A.

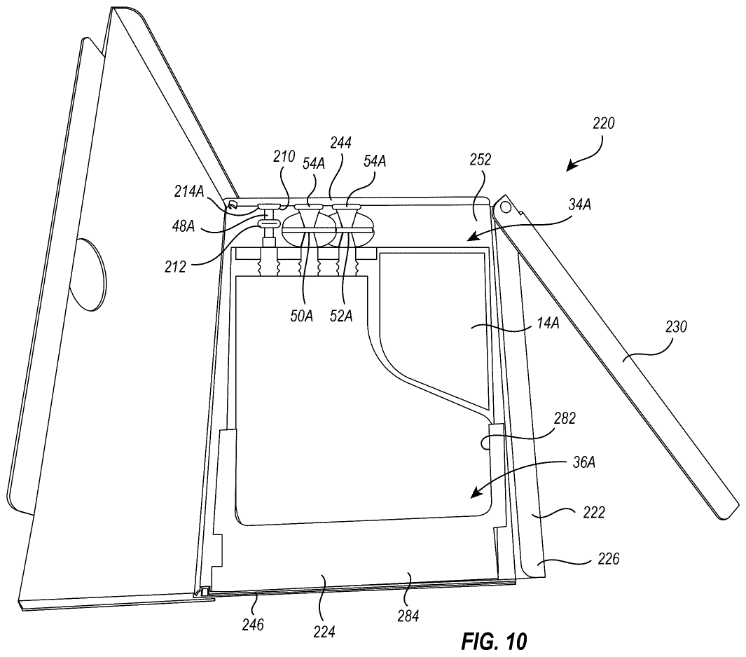

Turing to FIG. 10, during use, lower end 36A of product bag assembly 14A can be received within groove 282 of stabilizer 224 so that they are coupled together. The assembled product bag assembly 14A and stabilizer 224 are then received within cavity 252 so that tab 284/stabilizer 224 is disposed on or adjacent to second lip 246 of base 226 and terminal end 54A of ports 50A and 52A and inlet tube 48A are also disposed within cavity against or adjacent to first lip 244 of base 226. In other applications where bag assembly 14A is more properly sized to fit within cavity 252, stabilizer 224 can be eliminated.

It is noted that because the now terminal end of inlet tube 48A is at substantially the same height as terminal end 54A of ports 50A and 52A, ports 50A and 52A act to reinforce inlet tube 48A. That is, if cassette assembly 220 is inverted so that gravity is pushing upper end 34A of product bag assembly 14A against lip 244, the produced load or force would be distributed over inlet tube 48A and ports 50A and 52A, thereby limiting the movement of inlet tube 48A. Limiting the amount of movement of inlet tube 48A reduces the risk of failure to inlet tube 48A and weld seals 212 and 214A. Furthermore, by having inlet tube 48A the same height at ports 50A and 52A, product bag assembly 14A can be fit within cavity 252 of cassette 222 with less free space for movement of product bag assembly 14A therein, this further helps to limit the risk of damage to inlet tube 48A and seals 212 and 214A. In contrast, in conventional practice inlet tube 48A extends to at least weld seal 196 (FIG. 5) and thus projects considerably farther beyond terminal end 54A of ports 50A and 52A. Thus, in conventional practice, inlet tube 48A would need to be bent when being placed within cavity 252. Such bending of inlet tube 48A, however, can occasionally damage inlet tube 48 or the seal formed thereon, such as during placement with the cassette, during movement of the cassette and/or during freezing of the cassette. Alternatively, if inlet tube 48 is retained straight by enlarging the size the cassette, all of the load of the product bag assembly with suspension therein would independently be placed on inlet tube 48 if the cassette was inverted, thereby potentially damaging inlet tube 48 or the weld seal disposed therein and thereby jeopardizing the integrity of the suspension. The foregoing problems are avoided or minimized by reducing the length of inlet tube 48A and adding the second weld seal, as disclosed herein.

With product bag assembly 14A and stabilizer 224 disposed within cavity 252, cover 228 is moved to the closed position and locking arm 230 is moved to the locking position, thereby securing cassette 222 in the closed position. Cassette 222 can then be placed on a tray configured to receive cassettes 222 and placed within a cryogenic freezer, as is know in the art, for freezing the suspension therein. One example of a cryopreservation cassette that can be used as cassette 222 is CRYOPLUS Frame available from ThermoFisher Scientific. The above process can be repeated with product bag assemblies 14B and 14C.

It is appreciated that the disclosed methods and systems also have other benefits beyond those disclosed above. For example, attempts to freehand the formation of weld seals 210 and 212 (FIG. 8) resulted in poorly placed and poorly formed weld seals having a higher rate of failure. In contrast, by using tube sealing system 80 and the methods disclosed herein, weld seals 210 and 212 are more accurately and consistently formed with proper shape, orientation, and spacing, thereby minimizing failure of weld seals 210 and 212 and maximizing preservation of the suspension.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for sealing a tube extending from a product bag used in cryogenic freezing, the system comprising:
a support having a top surface;
a bag guide secured to the support so as to upstand from the top surface, the bag guide including a boundary face;
a tube sealer at least partially upstanding from the top surface of the support, the tube sealer comprising a first sealing head and a second sealing head and being movable between an open position and a closed position; and
a spacer arm movable between a blocking position wherein the spacer arm is disposed between the bag guide and the tube sealer so that the spacer arm at least partially covers the boundary face and an unblocking position wherein the boundary face is openly exposed.

2. The system as recited in claim 1, further comprising a tube guide upstanding from the top surface of the support and being disposed on a side of the tube sealer opposite of the bag guide, wherein the tube guide is configured to securely support the tube when the product bag is disposed on the top surface against the boundary face of the bag guide.

3. The system as recited in claim 1, wherein the spacer arm is hingedly mounted to the support or the bag guide.

4. The system as recited in claim 1, wherein the spacer arm has a front face and an opposing back face with a thickness extending therebetween in a range between 0.2 cm and 1 cm.

5. The system as recited in claim 1, wherein the bag guide has an inside face with a U-shaped configuration, the inside face including the boundary face and a pair of opposing side faces extending from opposing ends of the boundary face.

6. The system as recited in claim 1, wherein the support comprises a tabletop having the top surface.

7. The system as recited in claim 1, wherein a portion of the tube sealer projects through an opening formed on the top surface of the support.

8. The system as recited in claim 1, wherein the tube sealer generates a radio frequency to seal the tube between the first sealing head and the second sealing head when the tube sealer is in the closed position.

9. The system as recited in claim 1, further comprising:
the support comprising a table, the table including:
a tabletop having the top surface, an opening passing through the tabletop;
a first leg assembly projecting from a first end of the tabletop;
a second leg assembly projecting from a second end of the tabletop; and
a brace extending between the first leg assembly and the second leg assembly; and
the tube sealer being secured to the brace and partially projecting through the opening on the tabletop.

10. The system as recited in claim 1, further comprising:
the first sealing head having a first engaging surface configured to engage with the tube, the first engaging surface being flush with the top surface of the support; and
the second sealing head having a second engaging surface that is configured to engage with the tube, the second engaging surface being vertically spaced above the first engaging surface.

11. A method for sealing a tube extending from a product bag used in cryogenic freezing, the method comprising:
positioning the product bag on a top surface of a support so that a portion of the product bag rests against a boundary face of a bag guide and the tube passes through a portion of a tube sealer when the tube sealer is in an open position;
moving the tube sealer to a closed position so as to form a first weld seal across the tube that seals the tube closed;
moving the product bag relative to the bag guide so that the product bag is at a second position on the top surface that is spaced apart from the boundary face;
positioning a spacer arm between the product bag and the boundary face; and
moving the tube sealer again to the closed position so as to form a second weld seal across the tube that further seals the tube closed.

12. The method as recited in claim 11, wherein prior to moving the tube sealer to the closed position, a portion of the tube is secured to a tube guide, the tube guide being disposed on the top surface of the support on a side of the tube sealer opposite of the bag guide.

13. The method as recited in claim 11, further comprising positioning the portion of the product bag against a face of the spacer arm prior to moving the tube sealer again to the closed position.

14. The method as recited in claim 11, wherein the product bag has an upper end with the tube and a spaced apart first port projecting therefrom, the first port terminating at a terminal end, the method further comprising the first weld seal being formed so that when the first port and the tube are each extending linearly from the product bag in parallel alignment, a plane extending through a lateral central axis of the first weld seal and disposed perpendicular to the tube is within 5 mm of the terminal end of the first port.

15. The method as recited in claim 14, further comprising cutting laterally across the first weld seal so as to sever a portion of the tube.

16. The method as recited in claim 11, wherein positioning the spacer arm comprises pivoting the spacer arm that is hingedly mounted on the bag guide.

17. The method as recited in claim 11, wherein the product bag has a compartment that houses a biological cell suspension.

18. The method as recited in claim 17, further comprising enclosing the product bag with a cryogenic freezer cassette.

19. A product bag assembly used in cryogenic freezing, the product bag assembly comprising:
a product bag bounding a compartment and extending between an upper end and an opposing lower end, a biological cell suspension being disposed within the compartment;
a tube extending from the upper end of the product bag, a first weld seal and a spaced apart second weld seal being formed on the tube so as to seal the tube closed; and
a first port projecting from the upper end of the product bag and terminating at a terminal end,
wherein when the first port and the tube are each extending linearly from the product bag in parallel alignment, a plane extending through a lateral central axis of the first weld seal and disposed perpendicular to the tube is within 5 mm of the terminal end of the first port.

20. The product bag assembly as recited in claim 19, further comprising a second port projecting from the upper end of the product bag and terminating at a terminal end, the second port being spaced apart from the first port, wherein when the first port, the second port, and the tube are each extending linearly from the product bag in parallel alignment, a plane extending through a lateral central axis of the first weld seal and disposed perpendicular to the tube is within 5 mm of the terminal end of the first port and within 5 mm of the terminal end of the second port.

21. The product bag assembly as recited in claim 20, wherein the first port and the second port each comprise a spike port.

22. The product bag assembly as recited in claim 19, wherein the second weld seal is formed on the tube between the first weld seal and the product bag.
